(19) United States
(12) Patent Application Publication
(10) Pub. No.: US 2025/0383348 A1
(43) Pub. Date: Dec. 18, 2025

(54) SESSILE DROP BIOSENSOR AND EXTRACELLULAR VESICLE DETECTION METHOD USING SAME

(71) Applicant: IUCF-HYU (INDUSTRY-UNIVERSITY COOPERATION FOUNDATION HANYANG UNIVERSITY, Seoul (KR)

(72) Inventors: Sungyoung CHOI, Seoul (KR); Eun Jung LEE, Seoul (KR)

(73) Assignee: IUCF-HYU (INDUSTRY-UNIVERSITY COOPERATION FOUNDATION HANYANG UNIVERSITY, Seoul (KR)

(21) Appl. No.: 18/833,224

(22) PCT Filed: Jan. 26, 2023
(86) PCT No.: PCT/KR2023/001189

§ 371 Date: Feb. 21, 2025

(30) Foreign Application Priority Data
Jan. 28, 2022 (KR) 10-2022-0013173
May 11, 2022 (KR) 10-2022-0057794
Jul. 7, 2022 (KR) 10-2022-0083573

(51) Int. Cl. G01N 33/543; G01N 33/574

(52) U.S. Cl. CPC G01N 33/54366; G01N 33/57484

(57) ABSTRACT

Proposed are a sessile droplet biosensor and an extracellular vesicle detection method using same, wherein the sessile droplet biosensor can easily and conveniently perform superbright staining of proteins or lipids in extracellular vesicles through a non-specific staining material, such as CFSE, without a complicated signal generation process and can concentrate extracellular vesicles to a high concentration at the edges of sessile droplets by internal flowing induced by non-uniform evaporation in the sessile droplets, thereby detecting extracellular vesicles with high sensitivity. Moreover, the extracellular vesicle detection method using the sessile droplet biosensor can be utilized for standard setting technology for various diseases, such as cancer diagnosis standard setting technology, by the analysis of extracellular vesicle staining signals, or an information providing method for the analysis of extracellular vesicle staining signals can be utilized for early diagnosis of various diseases such as cancer, evaluation of prognosis for treatment, and screening for carcinoma.

### TECHNICAL FIELD

Proposed is a sessile droplet biosensor incorporating a superbright staining method and a concentration method to achieve a high concentration. Additionally, proposed is a method of detecting extracellular vesicles using the same.

### BACKGROUND ART

Extracellular vesicles (EVs) in the body, distributed evenly in the blood, urine, and saliva of patients, tend to have a high level of diagnostic convenience by enabling diagnostic sampling to be conducted with ease and thus are in the limelight as a new target for cancer diagnosis. Accordingly, EVs or exosomes tend to retain the properties of primary cells, are present in relatively high concentrations in the blood, and thus are actively used for early diagnosis of cancer and evaluation of treatment prognosis. For example, research is in active progress on immunodiagnostics to analyze the surface proteins of EVs or next-generation sequencing (NGS) of RNA and DNA in vesicles.

However, to analyze EVs and the like in the blood, interference resulting from large amounts of non-target substances (lipids and proteins) present in the blood must be removed, which is problematic. Although this has led to the development of methods such as ultracentrifugation and ExoQuick to purify EVs and the like, there has been a problem in that such sample preparation processes result in longer analysis time, making rapid diagnosis challenging.

In the meantime, another way to rule out the effect of non-target substances in the blood is to dilute the blood, but there has been a problem in that such excessive dilution leads to a decrease in the concentration of a target in a sample, making analysis challenging.

Therefore, there has been a demand for developing EV detection technology enabling targeted analysis with high sensitivity even when subjecting liquid samples to a 100-fold or greater dilution for rapid diagnosis.

### DISCLOSURE

### Technical Problem

Objectives to solve the problems described above are as follows.

One objective is to provide a sessile droplet biosensor integrating a superbright staining method and a concentration method to achieve a high concentration for detecting EVs with high sensitivity, and another objective is to provide a method of detecting EVs using the same.

### Technical Solution

A sessile droplet biosensor, according to a first aspect of the present disclosure, is characterized by including: a substrate; a functional base positioned on the substrate and including at least one pattern; and a bioreceptor positioned on the pattern and specifically binding to stained EVs, wherein a sessile droplet containing the EVs is formed on the pattern to have a predetermined contact angle with respect to the pattern, and an internal flow of the sessile droplet causes the EVs to migrate to the edge of the sessile droplet and specifically bind to the bioreceptor.

The contact angle may be in the range of 10° to 55°.

The pattern may be a perforation pattern made up of holes formed in the functional base or an uncoated pattern that is a region except for a region coated with a hydrophobic material on the substrate.

The pattern may have a maximum diameter in the range of 4 mm to 10 mm.

The EVs may be stained by a method that proteins or lipids in the EVs bind to a staining material.

The EVs may be isolated from at least one type of patient selected from the group consisting of a cancer patient, a brain disease patient, and a cardiovascular disease patient.

The bioreceptor may be at least one selected from the group consisting of an antibody, an aptamer, a nucleic acid, DNA, RNA, a biomimetic, a protein, an organic compound, and a polymer that specifically bind to the EVs.

A method of detecting EVs, according to a second aspect of the present disclosure, is characterized by including the following steps: staining a sample containing EVs; forming a sessile droplet containing the sample on a pattern of a sessile droplet biosensor; incubating the sessile droplet under a predetermined humidity condition, so that the EVs specifically bind to a bioreceptor positioned on the pattern; and detecting staining signals of the EVs specifically bound to the bioreceptor, wherein the sessile droplet is formed on the pattern to have a predetermined contact angle with respect to the pattern, and an internal flow of the sessile droplet causes the EVs to migrate to the edge of the sessile droplet and specifically bind to the bioreceptor.

The contact angle may be in the range of 10° to 55°.

The staining of the sample may be performed by binding proteins or lipids in the EVs to a staining material.

The staining of the sample may be performed for 30 minutes to 120 minutes.

Under the predetermined humidity condition for the incubation, a relative humidity may be in the range of 20% to 90%.

The incubation may be performed in a temperature range of 20° C. to 40° C.

The incubation may be performed for 85 minutes to 95 minutes.

A method of analyzing staining signals of EVs, according to a third aspect of the present disclosure, includes the following steps: obtaining a healthy domain and a cancer domain using a first result obtained through a quadratic discriminant analysis (QDA) classification algorithm from staining signals of EVs detected by the method of detecting the EVs; and

obtaining a specific cancer domain using a second result obtained through a multiclass QDA (MultiQDA) classification algorithm from staining signals of EVs in the cancer domain. using the first result obtained through QDA performed on the normalized data.

The step of obtaining the healthy and cancer domains may include the following steps: obtaining normalized data through principal component analysis (PCA) performed on the staining signals of the EVs; and obtaining the healthy and cancer domains using the first result obtained through QDA performed on the normalized data.

The step of obtaining the specific cancer domain may include: additionally obtaining normalized data through additional PCA performed on the staining signals of the EVs in the cancer domain; and obtaining the specific cancer domain using the second result obtained through MultiQDA performed on the additionally obtained normalized data.

The specific cancer domain may be a domain of at least one type of patient group selected from the group consisting of a lung cancer patient group, a liver cancer patient group, a breast cancer patient group, a colon cancer patient group, and a prostate cancer patient group.

A method of providing information to analyze staining signals of EVs, according to a fourth aspect of the present disclosure, is characterized by including the following steps: obtaining staining signals of EVs in the method of analyzing the staining signals of the EVs by detecting a biological sample of an individual in need thereof using the method of detecting the EVs; determining whether a first result obtained through a QDA classification algorithm from the staining signals of the EVs falls within a cancer domain; and determining a carcinoma using a second result obtained through a MultiQDA classification algorithm from the staining signals of the EVs when the first result falls within the cancer domain.

### Advantageous Effects

A sessile droplet biosensor, according to the present disclosure, can easily and conveniently perform superbright staining of proteins or lipids in EVs using a non-specific staining material without involving complicated signal generation process and can concentrate EVs to a high concentration at the edge of a sessile droplet by an internal flow induced by non-uniform evaporation in the sessile droplet, thereby detecting EVs with high sensitivity.

Accordingly, the method of detecting EVs using the sessile droplet biosensor can be applied to technologies for establishing criteria for various diseases, such as technology for establishing diagnostic criteria for cancer, by analyzing staining signals of EVS, or a method of providing information to analyze staining signals of EVs can be applied to early diagnosis of various diseases such as cancer, evaluation of treatment prognosis, and carcinoma screening, which is advantageous.

### DESCRIPTION OF DRAWINGS

FIG. 1 shows actual images of a sessile droplet biosensor fabricated according to Preparation Example 1;

FIG. 2 is a schematic diagram of the bottom of a sessile droplet in a sessile droplet biosensor divided into five zones (z1 to z5) according to one embodiment;

FIG. 3 is a schematic diagram illustrating image processing according to one embodiment;

FIG. 4 shows fluorescence area per unit area (1 mm²) as a function of incubation time when using an anti-epithelial cell adhesion molecule (EpCAM) antibody (anti-EpCAM) as a bioreceptor in a sessile droplet biosensor (EV-in-a-sessile-droplet; eSD) according to Preparation Example 1 and staining (5-(and-6)-carboxyfluorescein diacetate, succinimidyl ester (CFSE)) extracellular vesicles (Michigan Cancer Foundation-7 (MCF7) EVs) to detect staining signals (fluorescence signals), as a graph;

FIG. 5A shows nanoparticle tracking analysis (NTA) results of MCF7 EVs according to Preparation Example 2, as a graph;

FIG. 5B shows scanning electron microscope (SEM) images of specifically bound MCF7 EVs when using an anti-EpCAM as a bioreceptor in a sessile droplet biosensor (eSD) according to Preparation Example 1 and when using a control (IgG control);

FIG. 6 is a diagram schematically illustrating detecting EVs by varying the size of sessile droplets (20 μL and 50 μL) in a sessile droplet biosensor (eSD) according to Preparation Example 1 or using a standard micro-well according to Comparative Preparation Example 1;

FIG. 7A shows results obtained using an anti-EpCAM as a bioreceptor in a sessile droplet biosensor (eSD) according to Preparation Example 1 or in a standard micro-well according to Comparative Preparation Example 1 to detect MCF7 EVs, as graphs;

FIG. 7B shows MCF7 EV detection results obtained using a sessile droplet biosensor (eSD) according to Preparation Example 1 or a standard micro-well (using an anti-EpCAM as a bioreceptor) according to Comparative Preparation Example 1, as a graph of fluorescence area per unit area (1 mm²) as a function of bottom zones (z1 to z5) of a sessile droplet;

FIGS. 8A and 8B show sessile droplet images and schematic diagrams of internal flows depending on the size and contact angle of sessile droplets (20 μL, 55°; FIG. 8A) (50 μL, 95°; FIG. 8B) in a sessile droplet biosensor (eSD) according to Preparation Example 1 (top), and images showing the line lengths of fluorescent particles as a function of bottom zones (z1, z3, and z5) of the sessile droplet (bottom);

FIG. 9 shows fluorescent particle velocity as a function of bottom zones of a sessile droplet depending on the size and contact angle of a sessile droplet in a sessile droplet biosensor (eSD) according to Preparation Example 1, as a graph;

FIG. 10A shows fluorescence area per unit area (1 mm²) measured in a sessile droplet biosensor according to Preparation Example 1 or in a standard micro-well according to Comparative Preparation Example 1, as a graph as a function of incubation time;

FIG. 10B shows fluorescence area per unit area (1 mm²) measured in a sessile droplet biosensor according to Preparation Example 1 or in a standard micro-well according to Comparative Preparation Example 1, as a graph as a function of EV (MCF7 EV) concentration;

FIG. 11 shows results obtained using sessile droplet biosensors (eSD) including each bioreceptor (antibodies against anti-EpCAM, anti-CD147, anti-CD9, and anti-PSMA) to detect each cancer cell line-derived EV (MCF7 EVs, human colorectal tumor 116 (HCT116) EVs, lymph node carcinoma of the prostate (LNCaP) EVs, and hepatocellular carcinoma G2 (HepG2)), as graphs;

FIG. 12 is a comparison diagram of a staining signal heatmap of cancer cell line-derived EVs derived from a sessile droplet biosensor (eSD) with a signal heatmap of cell lines derived from flow cytometry (FCM);

FIG. 13 is a schematic diagram for multiplex detection of plasma-derived EVs, according to Example 6;

FIG. 14A shows results obtained using sessile droplet biosensors (eSD) including each bioreceptor (antibodies against EpCAM, CD24, and CD9) to detect each EV derived from healthy individuals and cancer patients (liver, colon, lung, breast, and prostate cancers), as graphs;

FIG. 14B shows results obtained using sessile droplet biosensors (eSD) including each bioreceptor (antibodies against CD147, epidermal growth factor receptor (EGFR), alpha-fetoprotein (AFP), and PSMA) to detect each EV derived from healthy individuals and cancer patients (liver, colon, lung, breast, and prostate cancers), as graphs;

FIG. 14C shows signal heatmaps of EVs derived from the plasma of cancer patients and healthy individuals, derived from a sessile droplet biosensor (eSD);

FIG. 15A shows NTA results for plasma samples of healthy individuals (control) and cancer patients, as a graph;

FIG. 15B shows scatter plots of individual staining signal levels (including unweighted sum) of cancer patients compared to staining signal levels of healthy individuals (control), derived from sessile droplet biosensors (eSD) with each bioreceptor;

FIG. 16 is a flowchart of a cancer classification algorithm according to Example 6;

FIG. 17 shows classification into a healthy group and a cancer group by applying data normalized by PCA to QDA, as a graph;

FIG. 18A shows MultiQDA results classified into specific cancer type groups using three principal components, as a graph;

FIG. 18B is a confusion matrix of cancer classification results classified through a MultiQDA classification algorithm;

FIG. 19A is a flowchart of a cancer classification algorithm leaving out PCA;

FIG. 19B is a confusion matrix of cancer classification results classified through a cancer classification algorithm leaving out data normalization through PCA;

FIG. 20A is a flowchart of a cancer classification algorithm using linear discriminant analysis (LDA) instead of QDA; and

FIG. 20B is a confusion matrix of cancer classification results classified through a cancer classification algorithm leaving out LDA.

### MODE FOR INVENTION

The above objectives, and other objectives, features, and advantages of the present disclosure will be readily understood from the following preferred embodiments associated with the accompanying drawings. However, the present disclosure is not limited to the embodiments described herein and may be embodied in other forms. The embodiments described herein are provided so that the disclosure can be made thorough and complete and that the technical spirit of the present disclosure can be fully conveyed to those skilled in the art.

Throughout the drawings, like elements are denoted by like reference numerals. In the accompanying drawings, the dimensions of the structures are larger than actual sizes for clarity of the present disclosure. Terms used herein, “first”, “second”, and the like, may be used to describe various components, but these components are not to be construed as being limited to these terms. These terms are used only for the purpose of distinguishing one component from another component. For example, without departing from the scope of the present disclosure, a first component may be referred to as a second component, and a second component may be also referred to as a first component. The singular expression includes the plural expression unless the context clearly indicates otherwise.

It will be further understood that the terms “comprises”, “includes”, or “has” when used herein specify the presence of stated features, integers, steps, operations, elements, components, and/or combinations thereof, but do not preclude the presence or addition of one or more other features, integers, steps, operations, elements, components, and/or combinations thereof. It will also be understood that when an element such as a layer, film, area, sheet, or the like is referred to as being “on” another element, not only the element may be directly on the other element, but also intervening elements may be present therebetween. Similarly, when an element such as a layer, film, area, sheet, or the like is referred to as being “under” another element, not only the element may be directly under the other element, but also intervening elements may be present therebetween.

Unless otherwise specified, all numbers, values, and/or expressions that represent amounts of components, reaction conditions, polymer compositions, and mixtures used herein are to be taken as approximations including a variety of uncertainties affecting measurement that inherently occur in obtaining such values, among others and should thus be understood to be modified by the term “about” in all cases. Furthermore, when a numerical range is disclosed herein, such a range is continuous and, unless otherwise indicated, includes all values from the minimum value of this range to the maximum value thereof. Moreover, when such a range refers to an integer, all integers including the minimum value to the maximum value are included, unless otherwise indicated.

As used herein, when a range is described for a variable, this variable will be understood as including all values within the stated range, including the stated endpoints of the range. For example, a range of “5 to 10” not only includes values of 5, 6, 7, 8, 9, and 10 but also includes any subranges such as 6 to 10, 7 to 10, 6 to 9, and 7 to 9. It will be understood that this range includes any value between reasonable integers within the scope of the stated range, such as 5.5, 6.5, 7.5, 5.5 to 8.5, and 6.5 to 9. Additionally, for example, a range of “10% to 30%” not only includes values, such as 108, 118, 12%, and 13%, and all integers up to and including 30% but also includes any subranges such as 10% to 158, 12% to 18%, and 20% to 30%. It will be understood that this range includes any value between reasonable integers within the scope of the stated range, such as 10.5%, 15.5%, and 25.5%.

Existing methods, including ultracentrifugation and ExoQuick, to detect and analyze EVs or exosomes have had problems in that sample preparation processes and the like result in longer analysis time, making rapid diagnosis challenging. Additionally, in the case of blood dilution methods, there has been a problem in that during excessive dilution, the concentration of a target in a sample decreases, making analysis challenging.

Accordingly, the inventors of the present disclosure conducted extensive research to develop EV detection technology enabling targeted analysis with high sensitivity even when subjected to 100-fold or greater dilution for rapid detection and diagnosis of EVs. As a result of the research, the inventors of the present disclosure found that EVs were detectable with high sensitivity because proteins or lipids in EVs were super-brightly stainable conveniently using a non-specific staining material without involving a complicated signal generation process, and EVs were able to be concentrated to a high concentration at the edge of a sessile droplet by an internal flow induced by non-uniform evaporation in the sessile droplet, thereby completing the invention of a sessile droplet biosensor, a detection method using the same, and the like.

A sessile droplet biosensor, according to a first aspect of the present disclosure, is characterized by including: a substrate; a functional base positioned on the substrate and including at least one pattern; and a bioreceptor positioned on the pattern and specifically binding to stained EVs, wherein a sessile droplet containing the EVs is formed on the pattern to have a predetermined contact angle with respect to the pattern, and an internal flow of the sessile droplet causes the EVs to migrate to the edge of the sessile droplet and specifically bind to the bioreceptor.

As used herein, the term “contact angle” may be a predetermined angle between the outer surface of the functional base and the tangent of the sessile droplet being in contact with the outer surface of the functional base.

As used herein, the term “EVs” may refer to lipid bilayer-delimited particles that are naturally released from specific cells.

According to the embodiment, the EVs may be isolated from at least one type of patient selected from the group consisting of a cancer patient, a brain disease patient, and a cardiovascular disease patient. Preferably, the EVs are isolated from at least one type of patient with cancer, of all cancer patients, selected from the group consisting of breast cancer, colorectal cancer, prostate cancer, and liver cancer. Alternatively, the EVs are isolated from at least one type of patient with brain diseases, of all brain disease patients, selected from the group consisting of Alzheimer's disease and Parkinson's disease. Alternatively, the EVs are isolated from at least one type of patient with cardiovascular diseases, of all cardiovascular disease patients, selected from the group consisting of myocardial ischemia and arteriosclerosis. However, the EVs are not limited to specific patient-derived EVs.

According to the embodiment, the EVs may be at least one selected from the group consisting of exosomes, microvesicles, apoptotic bodies, and the like, depending on size and synthetic pathway, but are not limited to specific types of EVs.

The substrate, according to the present disclosure, capable of supporting the sessile droplet biosensor, may, for example, be one selected from the group consisting of silicon (Si), gallium arsenide (GaAs), glass, quartz, and a polymer, but is not limited to a substrate only containing specific materials.

The functional base, according to the present disclosure, is positioned on the substrate and includes at least one pattern. Any functional base capable of forming the bioreceptor on the pattern is used without particular limitation.

Specifically, the pattern included in the functional base may be a perforation pattern made up of holes formed in the functional base or an uncoated pattern that is a region except for a region coated with a hydrophobic material on the substrate. According to the embodiment, the functional base may include the perforation pattern made up of holes formed therein. In this case, the substrate may be coated with a positively charged layer to form the bioreceptor on the perforation pattern. In this case, the positively charged layer is characterized by being adsorbed to relatively negatively charged NeutrAvidin later, followed by specifically binding biotinylated antibodies serving as the bioreceptor to NeutrAvidin immobilized due to positive and negative charges, thus stably immobilizing the bioreceptor. For example, the positively charged layer may be at least one compound selected from the group consisting of 3-aminopropyl triethoxysilane (APTES) and N (beta-aminoethyl) gamma-aminopropylmethyldimethoxysilane (AEAPMDMS) and is preferably APTES. However, the method of immobilizing the bioreceptor is not limited to the methods described above and may be any methods employable by those skilled in the art related to the present disclosure to immobilize the bioreceptor (for example, 1-Ethyl-3-[3-dimethylaminopropyl]carbodiimide (EDC)-N-hydroxysulfosuccinimide (NHS) coupling or antibody-Protein A/G binding).

According to the embodiment, the functional base, including the perforation pattern, may be at least one film selected from the group consisting of a polydimethylsiloxane (PDMS) film, a poly(methyl methacrylate) (PMMA) film, a poly D, L-lactic-co-glycolic acid (PLGA) film, and a silicone-based film. Preferably, the functional base is a PDMS film that easily forms the sessile droplet while being easily shapeable to form the perforation pattern.

Additionally, according to the embodiment, the functional base may include the uncoated pattern that is a region except for a region coated with the hydrophobic material therein. Preferably, the uncoated pattern may refer to a region formed by coating a region other than a sessile droplet formation region with the hydrophobic material or the sessile droplet formation region formed without being coated with the hydrophobic material. In this case, the uncoated pattern region may be coated with a positively charged layer to form the bioreceptor. Then, a method of immobilizing the bioreceptor may be the same as the immobilizing method in the case of the perforation pattern.

According to the embodiment, the hydrophobic material to form the uncoated pattern may be at least one selected from the group consisting of hydrophobic materials including hydrophobic nanoparticles, fluoro-silane, and trimethylchlorosilane.

In the meantime, the shape of the uncoated pattern, according to the embodiment, may be circular, tetragonal, triangular, and star-like, and the contents related to the specific material of the positively charged layer and the like may be the same as those described in the perforation pattern.

The sessile droplet, according to the present disclosure, is formed on the pattern of the functional base to have a predetermined contact angle with respect to the pattern. Additionally, the EVs contained in the sessile droplet specifically bind to the bioreceptor formed on the pattern and thus are detectable. Specifically, depending on the size of the pattern and, preferably, the maximum diameter of the pattern, the EVs are detectable by adjusting the contact angle of the sessile droplet formed on the pattern, the cross-sectional area where the sessile droplet and the pattern make contact, or the volume of the sessile droplet formed on the pattern.

The pattern, according to the embodiment, may have a maximum diameter in the range of 4 mm to 10 mm. Without falling within the above range, when the maximum diameter is extremely small, the amount of EVs in the sessile droplet may be extremely small, resulting in increased detection limit and poor sensitivity. Additionally, the contact angle of the sessile droplet may increase compared to droplets having larger diameters based on the same droplet volume, resulting in a poor EV concentration effect. On the contrary, when the maximum diameter is extremely large, the contact angle of the sessile droplet becomes extremely small, resulting in active evaporation. Thus, there is a disadvantage in that when the droplet dries, a false positive is detectable due to the increased non-specific binding.

In the meantime, depending on the maximum diameter of the pattern according to the embodiment, the contact angle of the sessile droplet may be in the range of 10° to 55°, the cross-sectional area where the sessile droplet and the pattern make contact may be in the range of 10 mm² to 100 mm², and the volume of the sessile droplet formed on the pattern may be in the range of 30 μL to 0.5 mL.

The bioreceptor, according to the present disclosure, is formed on the pattern of the functional base. Any bioreceptor capable of specifically binding to the EVs in the sessile droplet may be used without limitation. Preferably, the bioreceptor is a biotinylated bioreceptor capable of specifically binding to NeutrAvidin adsorbed onto the pattern and, more preferably, is a biotinylated antibody.

The bioreceptor, according to the embodiment, may be at least one selected from the group consisting of an antibody, an aptamer, an enzyme, a nucleic acid, DNA, RNA, a cell, a biomimetic, a protein, an organic compound, and a polymer that specifically bind to the EVs.

The stained EVs, according to the present disclosure, may specifically bind to the bioreceptor and, preferably, are characterized in that proteins or lipids in the EVs bind to a staining material, thus enabling superbright staining.

According to the embodiment, the staining material to stain the EVs may be at least one selected from the group consisting of non-specific staining materials such as carboxyfluorescein diacetate (CFDA), CFSE, 1,1′-dioctadecyl-3,3,3′,3′-tetramethylindocarbocyanine (DiI), and PKH, but is not limited to specific staining materials.

In other words, in the sessile droplet biosensor according to the present disclosure, the sessile droplet formed on the pattern of the functional base may cause localized non-uniform evaporation along the liquid-air interface due to the predetermined contact angle. The more acute the contact angle of the sessile droplet, the more likely the highest evaporation flux in the outermost zone of the sessile droplet may be.

In other words, the non-isothermal interface formed by the sessile droplet may cause a surface tension gradient that may lead to Marangoni flow. Specifically, the Marangoni effect may occur according to Equation 1.

$$Ma = - \frac{d σ}{dT} \frac{a Δ T}{μα} \qquad [ Equation 1 ]$$

In this case, σ refers to surface tension, T refers to temperature, a refers to a droplet radius on the contact line, μ refers to viscosity, and α refers to thermal diffusivity.

Referring to Equation 1 shown above, the more acute the contact angle of the sessile droplet, the more effectively the inside of the sessile droplet is stirred because the internal flow of the sessile droplet is induced radially from the center point of the sessile droplet to the periphery, enabling the EVs to migrate actively to the periphery. Therefore, the super-brightly stained EVs may be enabled to specifically bind to the bioreceptor through an antibody-antigen reaction by effectively concentrating the EVs to a high concentration in the cross section where the pattern and the sessile droplet make contact. Thus, there is an advantage of detecting EVs with high sensitivity using the sessile droplet biosensor according to the present disclosure.

A method of detecting EVs, according to a second aspect of the present disclosure, is characterized by including the following steps: S10 of staining a sample containing EVs; S20 of forming a sessile droplet containing the sample on a pattern of a sessile droplet biosensor; S30 of incubating the sessile droplet under a predetermined humidity condition, so that the EVs specifically bind to a bioreceptor positioned on the pattern; and S40 of detecting staining signals of the EVs specifically bound to the bioreceptor, wherein the sessile droplet is formed on the pattern to have a predetermined contact angle with respect to the pattern, and an internal flow of the sessile droplet causes the EVs to migrate to the edge of the sessile droplet and specifically bind to the bioreceptor.

In this case, the contents related to the method of detecting the EVs, which are the same as those described in the sessile droplet biosensor, may be omitted.

Step S10 is to super-brightly stain the EVs in the sample.

According to the embodiment, proteins or lipids in the EVs in the sample may be incubated for 30 minutes to 120 minutes, preferably for 60 minutes to 90 minutes, and bind to a non-specific stain, CFSE (cell-permeable and amine-reactive fluorescent dye), for staining. Without falling within the above range, when the staining time is extremely short, there is a disadvantage in that the staining intensity is low, so the detection signal is weak. When the staining time is extremely long, there is a disadvantage in that rapid diagnosis is challenging.

Step S20 is to form the sessile droplet containing the EVs stained according to Step S10, on the pattern of the sessile droplet biosensor.

According to the embodiment, the sessile droplet may be formed on the pattern of the sessile droplet biosensor by undergoing a pipetting step, preferably through a pipetting step using a liquid handler, from the sample containing the EVs stained according to Step S10.

Step S30 is to incubate the sessile droplet under the predetermined humidity condition so that the EVs in the sessile droplet, formed according to Step S20, specifically bind to the bioreceptor positioned on the pattern of the sessile droplet biosensor.

According to the embodiment, the EVs and the bioreceptor may be allowed to specifically bind through the process of incubating the sessile droplet positioned on the pattern of the sessile droplet biosensor under the following conditions: a relative humidity in the range of 20% to 90%, a temperature in the range of 20° C. to 40° C., and a duration in the range of 85 minutes to 95 minutes. Without falling within the above range, when the relative humidity is extremely low, there are disadvantages in that the evaporation of the sessile droplet occurs vigorously, causing the sessile droplet to dry out completely, and non-specific signals are detectable. When the relative humidity is extremely high, there is a disadvantage in that the internal flow of the sessile droplet is affected, leading to a poor concentration effect. Additionally, when the temperature is extremely low, the antigen-antibody reaction causing specific binding may occur slowly, resulting in poor sensitivity. When the temperature is extremely high, denaturation of the EVs may occur, adversely affecting the specific binding. Furthermore, when the incubation time is extremely short, there is a disadvantage in that the number of captured exosomes is small, resulting in poor detection sensitivity. When the incubation time is extremely long, there are disadvantages in that rapid diagnosis is challenging, and non-specific signals may increase.

Step S40 is to detect the staining signals of the EVs specifically bound to the bioreceptor.

According to the embodiment, the EVs specifically bound to the bioreceptor may be fluorescently imaged by taking fluorescence images. These images may then be processed to remove fluorescence background noise and unspecific aggregates, enabling effective fluorescence signals, that is, the staining signals, to be detected. Specifically, to remove fluorescence background noise from fluorescence images, fluorescence images may be normalized to a minimum fluorescence intensity value and then extracted as Gaussian-filtered images. Next, the total area of fluorophores (fluorescence-labeled EVs) within a critical size range may be measured, ultimately detecting fluorescence signals.

In other words, the method of detecting the EVs of the present disclosure enables the super-brightly stained EVs to specifically bind to the bioreceptor through a receptor-ligand reaction, preferably through the antigen-antibody reaction, by effectively concentrating the EVs to a high concentration while shifting the flow in the sessile droplet to the outermost zone (z5) in a state where the sessile droplet is controlled under predetermined conditions. Thus, there is an advantage in that the EVs are detectable with high sensitivity using the sessile droplet biosensor according to the present disclosure.

A method of analyzing staining signals of EVs, according to a third aspect of the present disclosure, includes the following steps: S10′ of obtaining a healthy domain and a cancer domain using a first result obtained through a QDA classification algorithm from staining signals of EVs detected by the method of detecting the EVs; and S20′ of obtaining a specific cancer domain using a second result obtained through a MultiQDA classification algorithm (multiclass cancer classification algorithm) from staining signals of EVs in the cancer domain. In this case, the contents related to the sessile droplet biosensor and the method of detecting the EVs may be omitted from the contents for describing the method of analyzing the staining signals of the EVs.

Step S10′ is to obtain the healthy and cancer domains using the staining signals of the EVs measured by the method of detecting the EVs. Specifically, Step S10′ includes the following steps: S11′ of obtaining normalized data by separately detecting EVs derived from a healthy group and a cancer group by the method of detecting the EVs and then separately performing PCA on the staining signals of the EVs detected; and S12′ of obtaining the healthy and cancer domains using the first result obtained through QDA performed on the normalized data.

In Step S11′, PCA may be performed using the fitcdiscr( ) function of MATLAB (MathWorks Inc.) (with a normalization parameter γ of 1) for binary discrimination between a healthy sample and a cancer patient sample.

In Step S12′, the healthy and cancer domains may be obtained through QDA that combines the normalized data for the healthy sample or the cancer patient sample obtained through PCA in Step S11′.

Step S20′, which is to obtain the specific cancer domain from the cancer domain obtained through Step S10′, may specifically include a step of obtaining the specific cancer domain using the second result obtained through MultiQDA performed on the staining signals of the EVs in the cancer domain.

In other words, in Step S20′, the specific cancer domain may be obtained using the second result obtained through MultiQDA performed on cancer patient data.

According to the embodiment, the specific cancer domain obtained through MultiQDA may be a domain of at least one type of patient group selected from the group consisting of a lung cancer patient group, a liver cancer patient group, a breast cancer patient group, a colon cancer patient group, and a prostate cancer patient group.

Preferably, the method of analyzing the staining signals of the EVs, according to the present disclosure, refers to a method of establishing diagnostic criteria for cancer. However, the method of analyzing the staining signals of the EVs is not limited to the method of establishing the diagnostic criteria for cancer alone. In other words, samples of various diseases, such as brain diseases and cardiovascular diseases, instead of cancer patient samples may be used and applied to the method of analyzing the staining signals of the EVs, thus enabling the use thereof as a method of establishing diagnostic criteria for various diseases such as brain diseases and cardiovascular diseases.

A method of providing information to analyze staining signals of EVs, according to a fourth aspect of the present disclosure, includes the following steps: S10″ of obtaining staining signals of EVs in the method of analyzing the staining signals of the EVs by detecting a biological sample of an individual in need thereof using the method of detecting the EVs; S20″ of determining whether a first result obtained through a QDA classification algorithm from the staining signals of the EVs falls within a cancer domain; and S30″ of determining a carcinoma using a second result obtained through a MultiQDA classification algorithm from the staining signals of the EVs when the first result falls within the cancer domain. In this case, the contents related to the sessile droplet biosensor, the method of detecting the EVs, and the method of analyzing the staining signals of the EVs may be omitted from the contents for describing the method of providing the information to analyze the staining signals of the EVs.

Step S10″ is to obtain the staining signals of the EVs by detecting the biological sample of the individual, which is a random sample, using the method of detecting the EVs. In this case, the analysis of the staining signals of the EVs may be performed in the same manner as in the method of analyzing the staining signals of the EVs.

Step S20″ is to determine whether the first result obtained through the QDA classification algorithm from the staining signals of the unknown biological sample obtained through Step S10″ falls within a healthy domain or a cancer domain. Specifically, the staining signal classification algorithm may be performed in the same manner as in Step S10′ described above, thereby determining whether the first result obtained falls within the healthy domain or the cancer domain obtained through Step S10′ and ultimately determining whether the unknown biological sample is derived from a healthy individual or a cancer patient.

Step S30″ is to determine a carcinoma using the second result obtained through the MultiQDA classification algorithm from the staining signals of the unknown biological sample when the unknown biological sample is determined to fall within the cancer domain through Step S20″. Specifically, the MultiQDA classification algorithm may be performed in the same manner as in Step S20′ described above, thereby determining which specific cancer domain among the specific cancer domain obtained through Step S20′ the second result obtained falls within and ultimately determining which specific type of cancer patient the unknown biological sample is derived from.

Preferably, the method of providing the information to analyze the staining signals of the EVs, according to the present disclosure, refers to a method of providing information to diagnose cancer. However, the method of providing the staining signals of the EVs is not limited to the method of providing the information to diagnose cancer alone. In other words, when samples of various diseases, such as brain diseases and cardiovascular diseases, instead of cancer patient samples may be used and applied to the method of analyzing the staining signals of the EVs, diagnostic criteria for various diseases can be established by methods of establishing diagnostic criteria for brain diseases, cardiovascular diseases, and the like, thus enabling the use thereof as a method of providing information to diagnose various diseases such as brain diseases and cardiovascular diseases, on the basis of the diagnostic criteria established as above.

The present disclosure will be described in more detail through the following examples. The following examples are only examples to help the understanding of the present disclosure, and the scope of the present disclosure is not limited thereto.

### Preparation Example 1—Fabrication of Sessile Droplet Biosensor

A specific method of fabricating a sessile droplet biosensor (EV-in-a-sessile-droplet; eSD) is as follows. First, a glass slide was prepared as a substrate. Then, the glass slide was coated with APTES, an amino silane compound, to coat an upper portion of the substrate with a positively charged layer. Specifically, the glass slide, serving as the substrate, was subjected to oxygen plasma treatment, treated with a 4% APTES solution, and then incubated, thereby coating the upper portion of the glass slide with APTES.

Next, the substrate coated with the positively charged layer was washed four times with ethanol (99%) and dried. Subsequently, a PDMS film (5-mm perforations) (0.25-mm thick) including a perforation pattern and serving as a functional base was prepared and positioned on the dried substrate. The perforation pattern was then filled with 200 μg/mL of NeutrAvidin (Thermo Fisher Scientific Corp., USA) and left at 25° C. for one hour. After washing the resulting product with filtered Dulbecco's phosphate-buffered saline (DPBS), each perforation pattern containing NeutrAvidin was filled with 10 μg/mL of a biotinylated antibody serving as a bioreceptor and left at 25° C. for two hours. In this case, specific biotinylated antibodies shown in Table 1 below were used.

TABLE 1

| Application | Target | Clone | Vendor | Catalog No. |
|---|---|---|---|---|
| eSD | CD9 (biotin) | MEM-61 | Invitrogen | MA1-19485 |
| (biotin) | CD24 (biotin) | eBioSN3 | eBioscience | 13-0247-82 |
| | | (SN3 A5-2H10) | | |
| | EpCAM (biotin) | 1B7 | eBioscience | 13-9326-82 |
| | CD147 (biotin) | MEM-M6/1 | Abcam | ab21898 |
| | EGFR (biotin) | 111.6 | Invitrogen | MA5-13266 |
| | AFP (biotin) | 1E8 | eBioscience | 13-9499-82 |
| | PSMA (biotin) | LNI-17 | Biolegend | 342510 |
| | IgG (biotin) | G18-145 | BD Biosciences | 555785 |
| FACS | CD9 (FITC) | MEM-61 | Invitrogen | MA1-19557 |
| | EpCAM (FITC) | EBA-1 | BD Biosciences | 347197 |
| | CD147 (FITC) | HIM6 | BD Biosciences | 555962 |
| | PSMA (APC) | LNI-17 | Biolegend | 342508 |
| | IgG (FITC) | MOPC-21 | BD Biosciences | 555748 |
| | IgG (APC) | MOPC-21 | BD Biosciences | 550854 |

Next, after washing the resulting product with filtered DPBS, the potential for non-specific binding was blocked using a 5% bovine serum albumin (BSA) solution (Sigma-Aldrich, Inc., USA). Then, the resulting product was washed twice with DPBS containing 0.05% Tween-20, ultimately fabricating the sessile droplet biosensor.

In the meantime, to fabricate a further highly multiplexed sessile droplet biosensor, a sessile droplet biochip was fabricated by stereolithography-based three-dimensional (3D) printing (Asiga Corp., Australia).

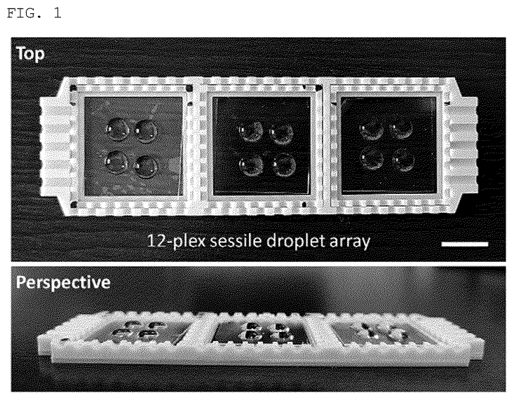

FIG. 1 shows actual images of the sessile droplet biosensor fabricated according to Preparation Example 1. Referring to FIG. 1, a two-dimensional droplet array may be used to arrange multiple (12) sessile droplets in parallel, so there is an advantage in that EVs are detectable through multiplex detection.

### Preparation Example 2—Preparation of Cancer Cell Line-Derived EVs

In a Roswell Park Memorial Institute (RPMI)-1640 medium (Welgene, Inc., Korea) supplemented with 10% (v/v) exosome-free fetal bovine serum (FBS) (System Bioscience, Inc., USA) and a 1% (v/v) penicillin-streptomycin solution (Welgene), human cancer cell lines (MCF7, HCT116, LNCaP, and HepG2) obtained from the Korean Cell Line Bank (Korea) were cultured at a temperature of 37° C. under a condition of humidified atmosphere containing 5% CO₂.

Supernatants (containing EVs) of the cell lines cultured in such a manner were obtained after three days of culturing and then centrifuged at 300 g and 2,000 g for 10 minutes to remove cancer cells and cancer cell fragments. Next, an additional spin-down was performed at 10,000 g for 30 minutes to remove large vesicles.

Then, after the spin-down, each supernatant was filtered through a 0.22 μm membrane filter (Millipore Corp., USA) and ultracentrifuged at 200,000 g for 80 minutes to form the EVs into pellets.

The EVs formed into the pellets were washed with DPBS at 200,000 g for 80 minutes and re-suspended in DPBS.

In the meantime, the size and concentration of the EVs collected through the re-suspension were measured using a nanoparticle tracking analyzer (Particle Metrix GmbH, Germany).

### Preparation Example 3—Preparation of Clinical Sample-Derived EVs

Plasma samples from healthy individuals and patients were collected from the National Biobank of Korea in accordance with a protocol (IRB #. HYUIRB-202102-007) approved by the Kyung Hee University Institutional Review Board. Information on pathologic diagnosis (Stage 3 or 4 cancer) was provided for all plasma samples (n=24).

To analyze EVs (eSD assay) using the sessile droplet biosensor, frozen plasma samples were thawed at room temperature, subjected to 100-fold dilution with DPBS to minimize the matrix effect of the plasma before analysis, and filtered through a 0.22 μm membrane filter (Millipore), thereby preparing EVs derived from plasma, serving as the clinical sample.

In the meantime, for NTA, 50 μL of the plasma was subjected to 100-fold dilution with DPBS and then centrifuged at 100,000 g for 30 minutes. The centrifuged solution was then filtered through a 0.22 μm membrane filter (Millipore) and ultracentrifuged at 200,000 g for 80 minutes to form the EVs into pellets. Next, the EVs formed into the pellets were washed with DPBS at 200,000 g for 80 minutes and re-suspended in DPBS.

### Preparation Example 4—Staining and Incubation of EVs

Cancer cell line-derived EVs were prepared according to Preparation Example 2 using DPBS without involving dilution. On the other hand, plasma-derived EVs were prepared as in Preparation Example 3.

Then, at 25° C., solutions of the cancer cell line-derived and plasma-derived EVs with each volume of 500 μL were independently mixed with 6 μL of CFSE (cell-permeable and amine-reactive fluorescent dye) and incubated, thereby labeling and staining the EVs. After sufficiently staining the EVs through incubation for 60 minutes, each solution stained in such a manner was mixed with 37 μL of 30% BSA and 6 μL of a human fragment crystallizable (Fc) blocker (BD Bioscience) and incubated for 10 minutes, thereby blocking unbound dyes and reducing non-specific CFSE uptake on the EV surface.

Next, after pipetting the resulting solution onto the sessile droplet biosensor, the EVs were allowed to specifically bind to a bioreceptor through incubation for 90 minutes in an incubator under the following conditions: a temperature of 25° C. and a relative humidity of 70%.

Subsequently, after washing each sessile droplet twice with DPBS, the EVs specifically bound to the bioreceptor were fluorescently imaged.

### Preparation Example 5—Fluorescence Imaging Setup and Analysis

Using a monochrome scientific complementary metal oxide semiconductor (CMOS) camera (Andor Technology, Ltd., United Kingdom) mounted on a Nikon inverted fluorescence microscope equipped with a motorized stage, the EVs (Preparation Example 4) specifically bound to the bioreceptor were fluorescently imaged by taking images with an exposure time in the range of 300 to 500 ms. Specifically, using a 40× objective lens with a numerical aperture of 0.75, the fluorescence-labeled EVs by staining were detected and fluorescently imaged.

The fluorescence images were analyzed using an in-house developed MATLAB analysis code, thus measuring the total area of fluorophores (fluorescence-labeled EVs). For each experimental condition, fluorescence images of up to ten different areas were taken, and then the average of fluorescence signals (staining signals), excluding the maximum and minimum, was obtained.

After intensity normalization of the fluorescence signals, the spatial fluorescence-intensity distributions were displayed in a 3D surface plot format to further clearly visualize the fluorophores.

### Preparation Example 6-SEM Imaging

The presence of the EVs bound to the bioreceptor in the sessile droplet biosensor was confirmed by SEM measurement.

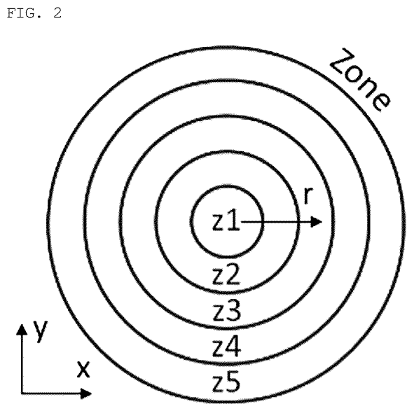

Specifically, to perform SEM imaging, 5×10⁶ EVs/μL of EVs (MCF7) contained in 20 μL of a sessile droplet were allowed to bind to an anti-EpCAM and an anti-IgG, serving as bioreceptors, and then the outermost region (z5), according to FIG. 2, was imaged.

Next, the EVs bound in such a manner were immobilized in 2% paraformaldehyde for 10 minutes and washed with DPBS and DI water.

Subsequently, after 12 hours of drying in a vacuum chamber, the bound EVs were coated with platinum for 40 seconds using an ion sputter coater (E-1010, Hitachi, Japan).

Next, SEM imaging was then performed using a field emission SEM (S-4700, Hitachi, Japan) at an operating voltage level of 10 kV.

### Preparation Example 7—FCM and EV Analysis (eSD Analysis)

FCM (Accuri C6; BD Biosciences, Inc., USA) was used to analyze the expression levels of cell-surface antigens from cancer cell lines.

Specifically, cells from cancer cell lines were washed twice with DPBS, stained at 4° C. for 40 minutes with fluorescein-conjugated antibodies against surface markers (that is, EpCAM, CD147, CD9, and PSMA), and additionally washed with DPBS for use in FCM analysis.

FCS Express (De Novo Software, USA) was used to analyze FCM data. Each fluorescence intensity was subtracted by the isotype control value thereof and then divided by the isotype value again to minimize cell size effects on the result, thereby calculating FCM signals.

The FCM signal intensity and the EV fluorescence signal (staining signal) intensity were divided by the 95th percentile value of all measured intensities for normalization, thus comparing a signal level difference between the two signals measured using different scales.

### Preparation Example 8-Cancer Classification Algorithm

In the present disclosure, data was normalized through PCA using a singular value decomposition method, followed by performing QDA.

In the first step, the fitcdiscr( ) function of MATLAB (MathWorks Inc.) (with a normalization parameter γ of 1) was used to perform binary discrimination between a healthy sample and a cancer patient sample.

The same MATLAB function was used for multiclass QDA in the second step.

Confusion matrix and statistical analysis in R were performed using the caretpackage based on the classification algorithm result.

### Preparation Example 9—Statistical Analysis

All experiments of the present disclosure were repeatedly conducted at least three times for consistent results and twice only in the case of clinical sample testing. Each measurement point was expressed as mean and standard deviation (S.D.) values.

Unless otherwise specified, the normalized intensity for the staining signals of the sessile droplet biosensor (eSD) against each marker in the cancer cell line sample or clinical sample was divided by the 95th percentile value of all measured intensities for calculation.

In the meantime, a direct comparison between the EV fluorescence signals of patients and healthy individuals (control) was performed using a non-parametric, two-tailed Mann-Whitney U-test (significance level of P<0.05).

The statistical analysis thereof was performed using GraphPad Prism 9 (GraphPad Software, Inc., USA).

Accuracy was defined as the probability of correctly classifying cancer. Sensitivity was defined as the probability of obtaining a positive result when the sample was derived from cancer cells. Additionally, specificity was defined as the probability that the sample was negative when derived from non-cancer cells.

Confidence intervals for sensitivity, specificity, and accuracy were calculated on the basis of a binomial distribution using the Clopper-Pearson exact method.

### Comparative Preparation Example 1-Fabrication of Standard Micro-Well

A micro-well was fabricated by punching a 1 mm-thick PDMS block using a 5 mm biopsy punch, bonding the resulting block to a cover glass through plasma treatment, and functionalizing the cover glass as in the above protocol.

### Example 1-EV Detection Method Using Sessile Droplet Biosensor

A sessile droplet biosensor was prepared according to Preparation Example 1.

Subsequently, cancer cell line-derived EVs and plasma-derived EVs were prepared according to Preparation Examples 2 and 3, respectively, and then fluorescently imaged as in Preparation Examples 4 and 5. Next, fluorescence signals (staining signals) were detected.

FIG. 2 is a schematic diagram of the bottom of the sessile droplet in the sessile droplet biosensor divided into five zones (z1 to z5) according to one embodiment. Referring to FIG. 2, Preparation Example 4, and Preparation Example 5, when dividing the bottom of the sessile droplet into five zones, EVs specifically bound to the bioreceptor in the outermost zone (z5) were fluorescently imaged by taking images.

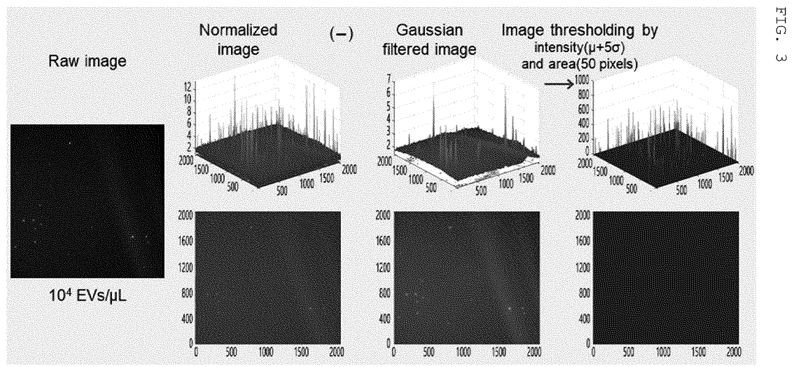

FIG. 3 is a schematic diagram illustrating image processing according to one embodiment. Referring to FIG. 3, Preparation Example 4, and Preparation Example 5, after the EVs specifically bound to the bioreceptor were fluorescently imaged, the images were processed to remove fluorescence background noise and unspecific aggregates, detecting effective fluorescence signals (staining signals). Specifically, to remove fluorescence background noise from fluorescence images, fluorescence images were normalized to a minimum fluorescence intensity value and then extracted as Gaussian-filtered images. Next, the total area of fluorophores (fluorescence-labeled EVs) within a critical size range was measured, ultimately detecting fluorescence signals.

In this case, for each experimental condition, fluorescence images of ten different areas were taken, and the average of fluorescence imaging, excluding the maximum and minimum, was obtained. Additionally, the spatial fluorescence-intensity distributions were displayed in a 3D surface plot format to further clearly visualize the fluorophores.

### Example 2—Review of EV Staining Conditions

Staining conditions required when detecting EVs using the sessile droplet biosensor, according to Example 1, were reviewed.

Specifically, EVs (MCF7 EVs) isolated from a medium in which human breast cancer cell line MCF7 was cultured according to Preparation Example 2 were used, and as the bioreceptor in the sessile droplet biosensor, an anti-EpCAM was used.

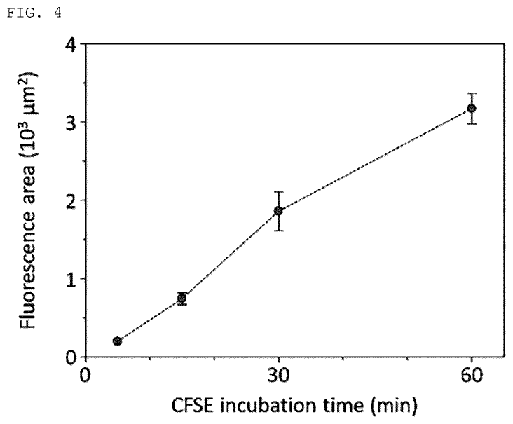

FIG. 4 shows fluorescence area per unit area (1 mm²) as a function of incubation time when using the anti-EpCAM as the bioreceptor in the sessile droplet biosensor (eSD) according to Preparation Example 1 and staining (CFSE) the EVs (MCF7 EVs) to detect staining signals (fluorescence signals), as a graph.

Referring to FIG. 4, it was confirmed that the fluorescence area increased significantly when the incubation time for the staining (CFSE) of the EVs (MCF7 EVs) was 30 minutes and continued to increase up to an incubation time of 60 minutes. This confirmed that an incubation time of 60 minutes or longer was sufficient for EV staining.

### Example 3—Review of EV Detection Specificity Using Sessile Droplet Biosensor

EVs were detected according to Example 1, using the sessile droplet biosensor.

Specifically, EVs (MCF7 EVs) isolated from a medium in which human breast cancer cell line MCF7 was cultured according to Preparation Example 2 were used.

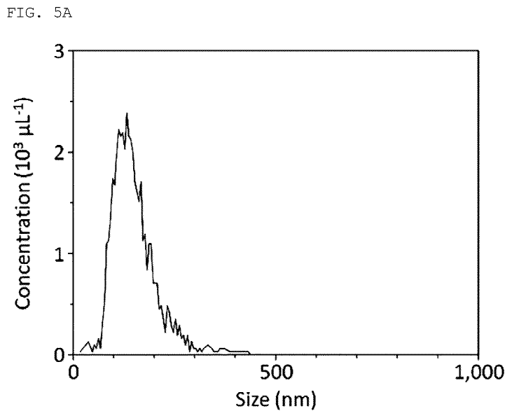

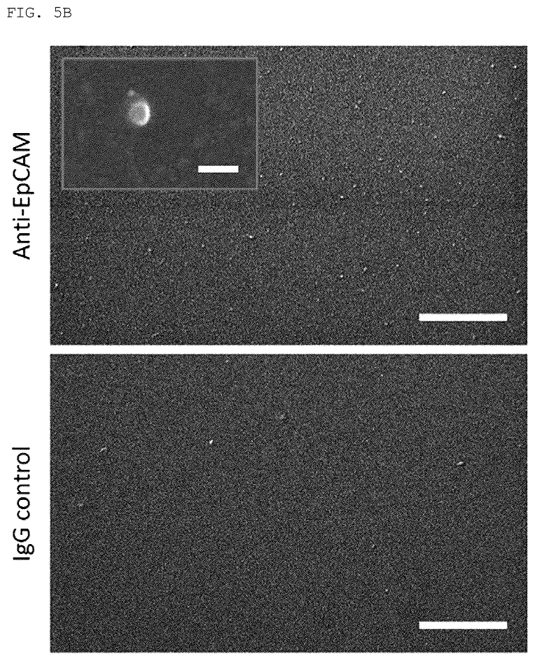

FIG. 5A shows NTA results of the MCF7 EVs according to Preparation Example 2, as a graph.

Referring to FIG. 5A, it was confirmed that the MCF7 EVs were most frequent at a size of 139.3 nm.

Additionally, as the bioreceptor in the sessile droplet biosensor, an anti-EpCAM and a control (IgG control) were separately used.

FIG. 5B shows SEM images of the specifically bound MCF7 EVs when using the anti-EpCAM as the bioreceptor in the sessile droplet biosensor (eSD) according to Preparation Example 1 and when using the control (IgG control).

Referring to FIG. 5B, as a result of the SEM imaging according to Preparation Example 6, it was confirmed that compared to the IgG control, the eSD including the anti-EpCAM bound to a much greater number of MCF7 EVs, showing high immunodetection specificity.

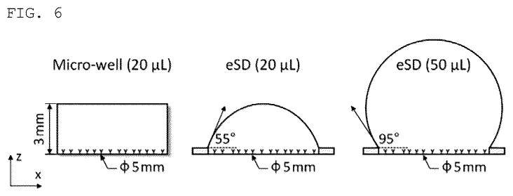

FIG. 6 is a diagram schematically illustrating detecting the EVs by varying the size of sessile droplets (20 μL and 50 μL) in the sessile droplet biosensor (eSD) according to Preparation Example 1 or using the standard micro-well according to Comparative Preparation Example 1.

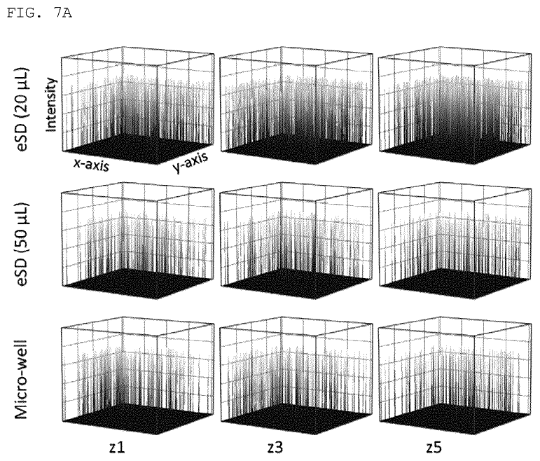

Referring to FIG. 6, the EVs were detected by varying the size of the sessile droplets (20 μL and 50 μL) in the sessile droplet biosensor (eSD) or using the standard micro-well. The results thereof are shown in FIGS. 7A and 7B.

Specifically, FIG. 7A shows results obtained using the anti-EpCAM as the bioreceptor in the sessile droplet biosensor (eSD) according to Preparation Example 1 or in the standard micro-well according to Comparative Preparation Example 1 to detect the MCF7 EVs, as graphs.

Additionally, FIG. 7B shows the MCF7 EV detection results obtained using the sessile droplet biosensor (eSD) according to Preparation Example 1 or the standard micro-well (using the anti-EpCAM as the bioreceptor) according to Comparative Preparation Example 1, as a graph of fluorescence area per unit area (1 mm²) as a function of bottom zones (z1 to z5) of the sessile droplet.

Referring to FIGS. 7A and 7B, when detecting the MCF7 EVs (105 EVs/μL) by varying the size of the sessile droplets (20 μL and 50 μL), it was confirmed that when the sessile droplet had a size of 20 μL, the nearer to the periphery of the droplet, the higher the fluorescence signals of the MCF7 EVs.

On the other hand, when the sessile droplet had a size of 50 μL or when the MCF7 EVs were detected in the micro-well (105 EVs/μL), it was confirmed that the fluorescence signals of the MCF7 EVs were relatively lower regardless of the bottom zone of the droplet.

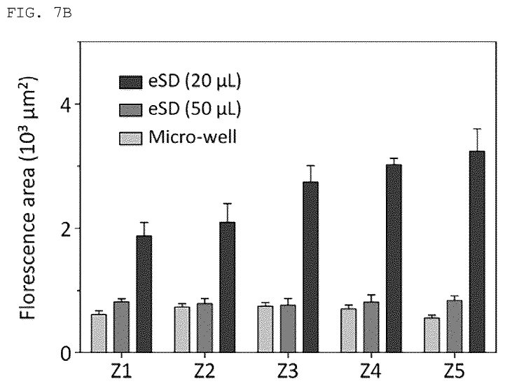

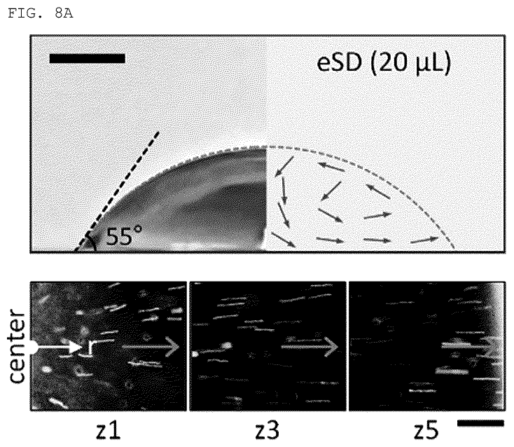

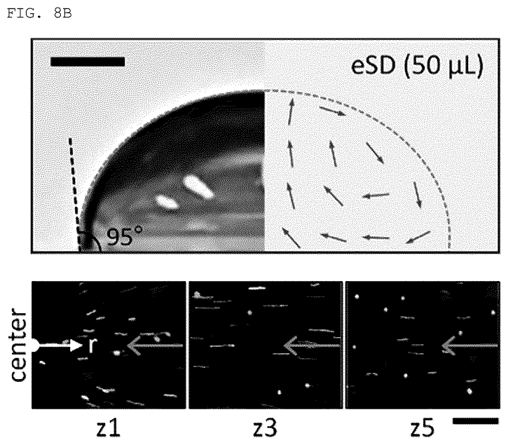

Additionally, during the fluorescence imaging setup according to Preparation Example 5, fluorescent 1-μm polystyrene particles (Thermo Fisher Scientific) were applied to the sessile droplet to perform particle image velocimetry, thereby measuring the respective flow velocities in the bottom zones of the sessile droplet. Specifically, the line length of the fluorescent particles (1-μm polystyrene particles) was divided by the exposure time for fluorescence imaging to measure velocity. The results thereof are shown in FIGS. 8A, 8B, and 9.

Specifically, FIGS. 8A and 8B show sessile droplet images and schematic diagrams of internal flows depending on the size and contact angle of the sessile droplets (20 μL, 55°; FIG. 8A) (50 μL, 95°; FIG. 8B) in the sessile droplet biosensor (eSD) according to Preparation Example 1 (top), and images showing the line lengths of the fluorescent particles as a function of bottom zones (z1, z3, and z5) of the sessile droplet (bottom).

Referring to FIGS. 8A and 8B, it was confirmed that the flow velocities measured in each bottom zone of the sessile droplet varied depending on the size and contact angle of the sessile droplet.

In other words, as the contact angle of the sessile droplet increases to 90° or higher, the position of the maximum evaporation rate shifts from the edge to the center point, confirming that the evaporation profile is non-uniform depending on the contact angle of the sessile droplet (or variable liquid-air interface). Additionally, it was confirmed that the Marangoni effect was induced in the sessile droplet formed in the sessile droplet biosensor.

FIG. 9 shows fluorescent particle velocity as a function of bottom zones of the sessile droplet depending on the size and contact angle of the sessile droplet in the sessile droplet biosensor (eSD) according to Preparation Example 1, as a graph.

Referring to FIG. 9, it was confirmed that the average radial velocity of the fluorescent particles at the bottom of the sessile droplet was 9.0 μm/s when the sessile droplet had a size of 20 μL, but was-9.9 μm/s when the sessile droplet had a size of 50 μL.

In summary, when the contact angle of the sessile droplet increases, the internal flow of the sessile droplet shifts from the edge to the center point, making it challenging to concentrate EVs efficiently in a specific sessile droplet bottom zone. On the contrary, as the contact angle of the sessile droplet decreases, the internal flow of the sessile droplet shifts to the contact line, that is, the edge of the sessile droplet, confirming that EVs are concentrated effectively in a specific sessile droplet bottom zone, that is the outermost zone (z5).

In particular, the sessile droplet biosensor, according to one embodiment, is advantageous in that when the contact angle of the sessile droplet is adjusted to be in the range of 10° to 55°, EVs are efficiently concentrated to the edge of the sessile droplet, facilitating EV detection.

### Example 4—Review of EV Detection Sensitivity Using Sessile Droplet Biosensor

The sessile droplet biosensor, according to Preparation Example 1, or the standard micro-well, according to Comparative Preparation Example 1, was prepared, followed by detecting EVs according to Example 1, to review detection sensitivity.

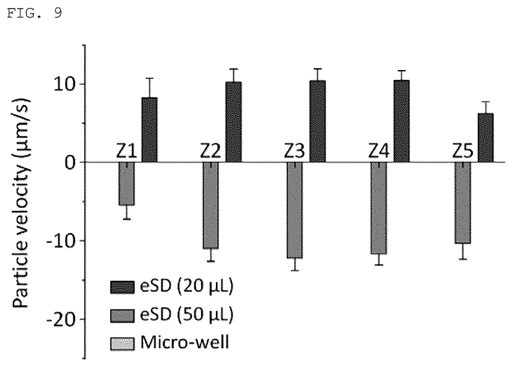

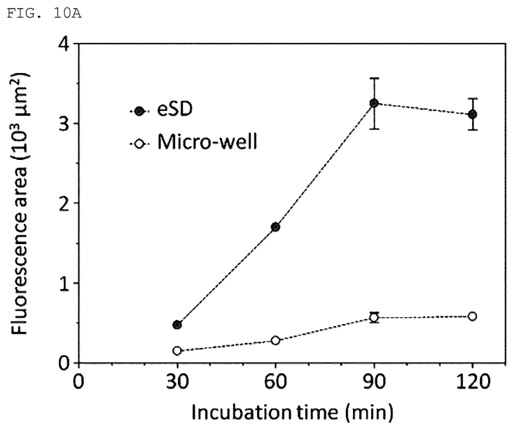

Specifically, an anti-EpCAM was prepared as the bioreceptor. In the meantime, MCF7 EVs were detected by using EVs (MCF7 EVs) isolated from a medium in which human breast cancer cell line MCF7 was cultured according to Preparation Example 2 with varying concentrations or by varying incubation time. The results thereof are shown in FIGS. 10A and 10B.

FIG. 10A shows fluorescence area per unit area (1 mm²) measured in the sessile droplet biosensor according to Preparation Example 1 or in the standard micro-well according to Comparative Preparation Example 1, as a graph as a function of incubation time.

Referring to FIG. 10A, it was confirmed that the EVs (MCF7 EVs) were the most effectively detectable when the incubation time for the binding of the antibody (anti-EpCAM), serving as the bioreceptor, to the EVs (MCF7 EVs) was less than 90 minutes.

FIG. 10B shows fluorescence area per unit area (1 mm²) measured in the sessile droplet biosensor according to Preparation Example 1 or in the standard micro-well according to Comparative Preparation Example 1, as a graph as a function of EV (MCF7 EV) concentration.

Referring to FIG. 10B, the limit of detection (LOD) value in the standard micro-well was 2103.2 EVs/μL, calculated from the value obtained by adding three times the standard deviation to the Blanck signal. On the contrary, the sessile droplet biosensor (eSD), according to Preparation Example 1, exhibited a reduced LOD value of 384.7 EVs/μL, which is comparable to the detection sensitivity of existing thermophoretic aptasensor (TAS) (3.3×10³ EVs/μL) or nano-herringbone (NB) chips (10 EVs/μL) in the art, confirming that the detection sensitivity was excellent.

In other words, the sessile droplet biosensor, according to one embodiment, is advantageous in that the detection sensitivity is excellent without involving existing complicated equipment setup nor requiring additional analysis such as later detection antibody labeling and enzymatic analysis.

### Example 5—Multiplex Detection of Cancer Cell Line-Derived EVs

Sessile droplet biosensors including each bioreceptor, anti-EpCAM, anti-CD147, anti-CD9, and anti-PSMA, were prepared according to Preparation Example 1. Then, multiplex detection of cancer cell line-derived EVs (EVs derived from human breast cancer cell line MCF7, colorectal carcinoma cell line HCT116, prostate adenocarcinoma cell line LNCaP, and Hepatocellular carcinoma cell line HepG2) was performed according to Example 1. The results thereof are shown in FIG. 11.

FIG. 11 shows results obtained using the sessile droplet biosensors (eSD) including each bioreceptor (antibodies against anti-EpCAM, anti-CD147, anti-CD9, and anti-PSMA) to detect each of the cancer cell line-derived EVs (MCF7 EVs, HCT116 EVs, LNCaP EVs, and HepG2), as graphs.

Referring to FIG. 11, when detecting cancer cell line-derived EVs by varying the type thereof in the sessile droplet biosensors including each bioreceptor, it was confirmed that different results were clearly obtainable.

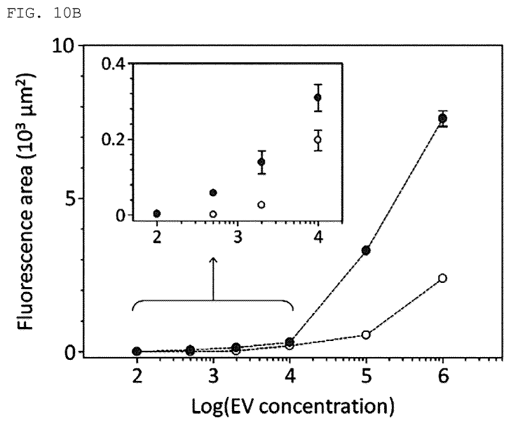

Additionally, to determine the correlation between the levels of cancer cell line-derived protein markers and the amount of EV subtypes, the levels of protein markers and the amount of EV subtypes were quantified through FCM and sessile droplet biosensor (eSD) analysis, respectively, according to Preparation Example 7. The results thereof are shown in FIG. 12.

FIG. 12 is a comparison diagram of a staining signal heatmap of the cancer cell line-derived EVs derived from the sessile droplet biosensor (eSD) with a signal heatmap of the cell lines derived from FCM.

Referring to FIG. 12, it was confirmed from the EV and cell line signals that the HCT116 EVs and cell lines showed high-level signals in both anti-EpCAM and anti-CD147, but the MCF7 EVs and cell lines showed a unique pattern of low-level signals, which was consistent with those in PSMA. In other words, it was confirmed that the staining signal pattern measured by the sessile droplet biosensor (eSD) was highly correlated with FCM.

### Example 6—Multiplex Detection of EVs Derived from Plasma of Healthy Individual and Cancer Patient

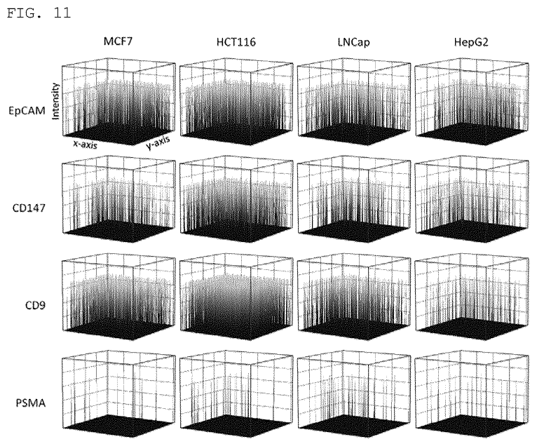

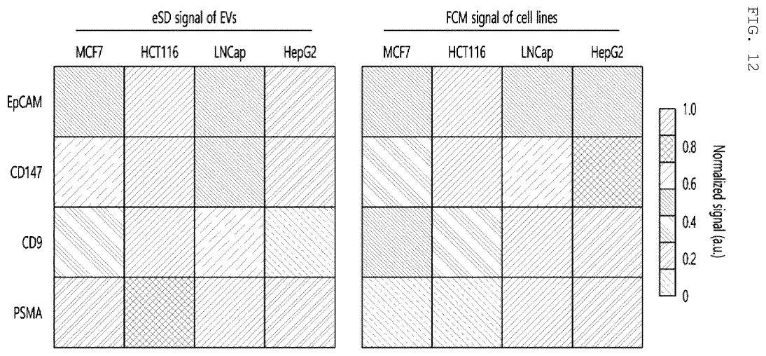

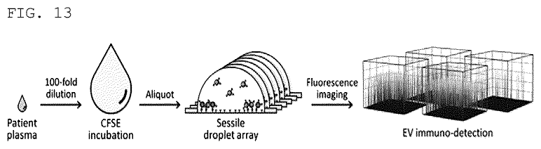

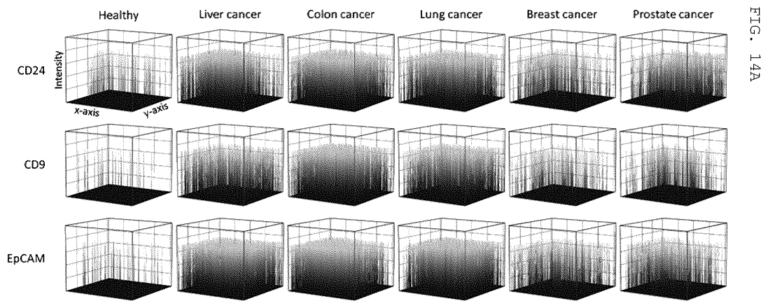

FIG. 13 is a schematic diagram for multiplex detection of plasma-derived EVs, according to Example 6. Referring to FIG. 13, sessile droplet biosensors (eSD) including each bioreceptor, antibodies against CD24, CD9, EpCAM, CD147, EGFR, AFP, and PSMA, were prepared according to Preparation Example 1. Additionally, EVs derived from the plasma of 20 cancer patients (liver, colon, lung, breast, and prostate cancers; n=4 for each cancer type) and four healthy individuals (control) were separately prepared according to Preparation Example 3. In this case, each level of the plasma-derived EVs ranged from 9.5×10⁶ EVs/μL to 6.6×10⁸ EVs/μL (which was high enough to perform sessile droplet biosensor (eSD) analysis even after 100-fold dilution). Then, the multiplex detection of the plasma-derived EVs prepared in such a manner was performed using the sessile droplet biosensor (eSD) prepared according to Example 1. The results thereof are shown in FIGS. 14A and 14B.

FIG. 14A shows results obtained using the sessile droplet biosensors (eSD) including each bioreceptor (antibodies against EpCAM, CD24, and CD9) to detect each EV derived from healthy individuals and cancer patients (liver, colon, lung, breast, and prostate cancers), as graphs.

FIG. 14B shows results obtained using the sessile droplet biosensors (eSD) including each bioreceptor (antibodies against CD147, EGFR, AFP, and PSMA) to detect each EV derived from healthy individuals and cancer patients (liver, colon, lung, breast, and prostate cancers), as graphs.

FIG. 14C shows signal heatmaps of the EVs derived from the plasma of cancer patients and healthy individuals, derived from the sessile droplet biosensor (eSD).

Referring to FIGS. 14A to 14C, it was confirmed that, as opposed to healthy individuals (control) with little or no signal, the signals derived from the sessile droplet biosensor (eSD) showed clearly different patterns by the types of cancer patients.

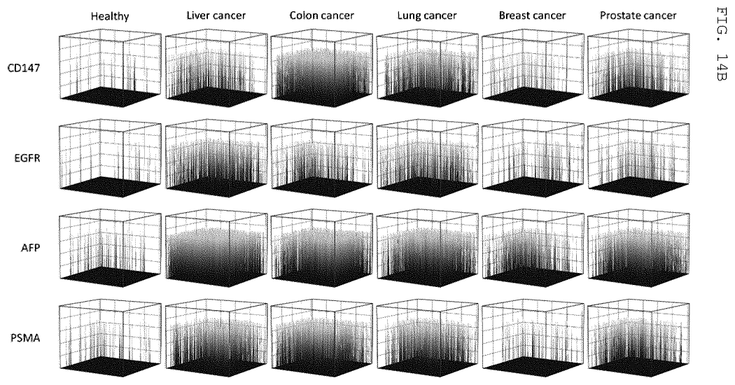

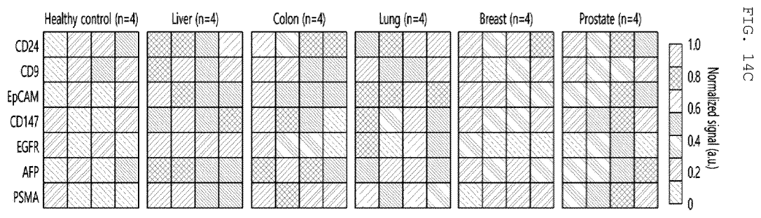

In the meantime, FIG. 15A shows NTA results for the plasma samples of healthy individuals (control) and cancer patients, as a graph, and FIG. 15B shows scatter plots of the individual staining signal levels (including unweighted sum) of cancer patients compared to the staining signal levels of healthy individuals (control), derived from the sessile droplet biosensors (eSD) with each bioreceptor. In this case, error bars mean±SD. Additionally, statistical comparisons were performed by a two-tailed Mann-Whitney U-test.

Referring to FIG. 15A, as a result of the NTA, it was confirmed that there was no significant difference in the EV concentration of the plasma of healthy individuals (control) and cancer patients. However, referring to FIG. 15B, it was confirmed that the individual staining signal levels (including unweighted sum) of cancer patients, derived from the sessile droplet biosensor (eSD), clearly increased when compared to the staining signal levels of healthy individuals (control).

In other words, whether a random plasma sample is derived from a cancer patient can be unambiguously identified and determined using the sessile droplet biosensor (eSD).

### Example 7—Analysis of Cancer Classification Algorithms for Cancer Type Classification

To classify specific cancer types from the plasma-derived EV detection analysis results of Example 5, a two-step cancer classification algorithm was analyzed.

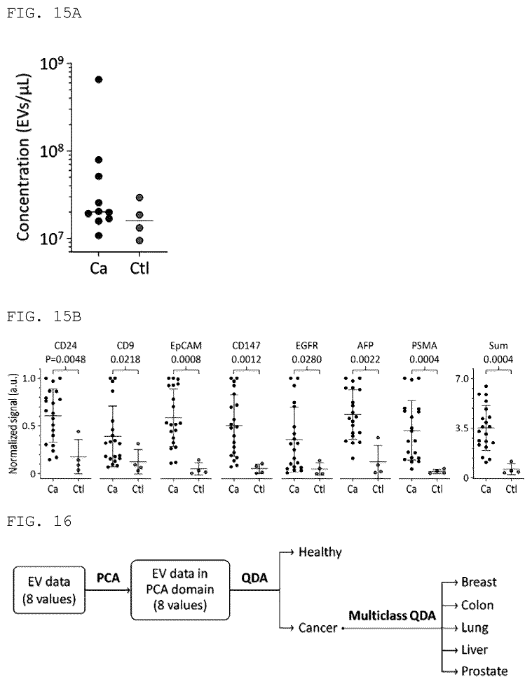

FIG. 16 is a flowchart of a cancer classification algorithm according to Example 6.

Referring to FIG. 16, in a first classification step, a healthy group and a cancer group were classified through a QDA classification algorithm according to Preparation Example 8.

Specifically, PCA was performed on the staining signals, indicating the data on the plasma-derived EV detection analysis result (raw data) from Example 5, for data normalization. In this case, PCA was performed as a pre-processing process not only for data normalization but also for visualization without dimensionality reduction. Then, QDA to plot two highly representative pieces of normalized data among data transformed by PCA and normalized on a graph was applied, classifying the healthy and cancer groups. The results are shown in FIG. 16.

FIG. 17 shows classification into the healthy and cancer groups by applying the data normalized by PCA to QDA, as a graph. In this case, parentheses represent variances captured in each principal component.

Referring to FIG. 17, the healthy group (n=4) was differentiated from the cancer group with 100% accuracy (95% CI: 86-100%), confirming that the healthy and cancer groups were clearly differentiated through the first classification algorithm from the staining signals, indicating the data on the plasma-derived EV detection analysis result (raw data), derived using the sessile droplet biosensor.

In a second classification step, through a MultiQDA classification algorithm, specific cancer type groups were classified from the cancer group having been classified through the first classification step.

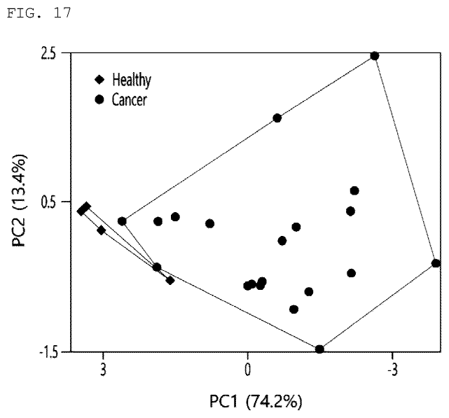

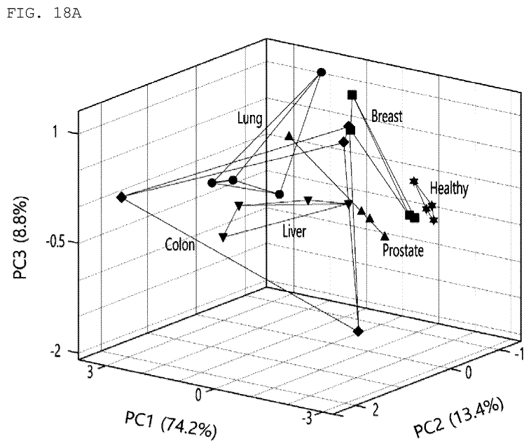

Specifically, the same algorithm as the classification algorithm according to Preparation Example was used. Additionally, to the cancer group having been classified through QDA to plot two highly representative pieces of normalized data, MultiQDA to plot one highly representative piece of normalized data by addition on a graph was applied, classifying the specific cancer type groups. The results thereof are shown in FIGS. 18A and 18B.

FIG. 18A shows the MultiQDA results classified into specific cancer type groups using three principal components, as a graph.

FIG. 18B is a confusion matrix of the cancer classification results classified through the MultiQDA classification algorithm.

Referring to FIG. 18A, it was confirmed that the specific cancer type groups were classified and identified through the MultiQDA classification algorithm. Additionally, through FIG. 18B, it was confirmed that due to excellent overall accuracy (95% CI: 75-100%) of 95%, the specific cancer type groups were highly likely to match the respective actual cancer types.

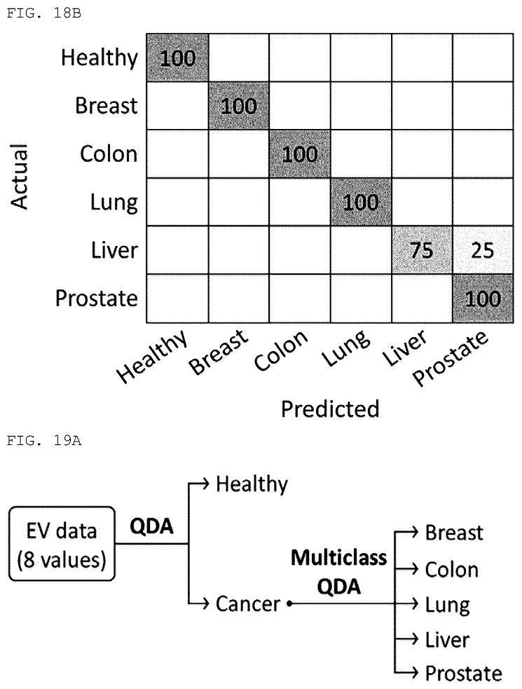

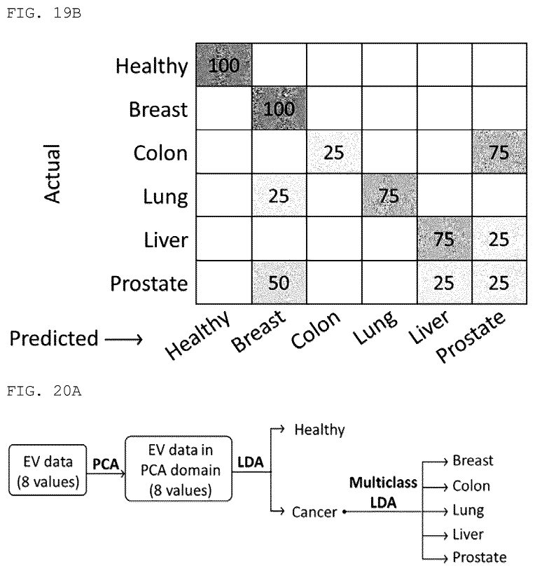

On the other hand, FIG. 19A is a flowchart of the cancer classification algorithm leaving out PCA, and FIG. 19B is a confusion matrix of the cancer classification results classified through a cancer classification algorithm leaving out data normalization through PCA.

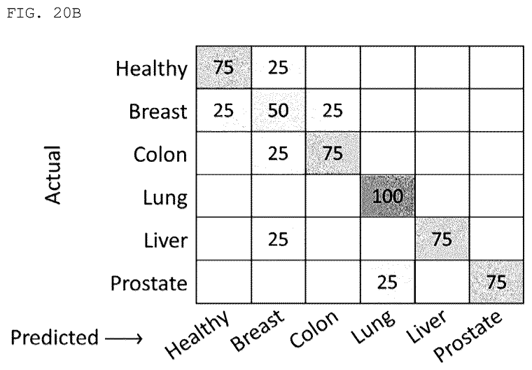

Additionally, FIG. 20A is a flowchart of the cancer classification algorithm using LDA instead of QDA, and FIG. 20B is a confusion matrix of the cancer classification results classified through a cancer classification algorithm leaving out LDA.

Referring to FIGS. 19A to 20B, when classifying the cancer types by leaving out PCA or replacing QDA with LDA from the cancer classification algorithm, the overall accuracy (95% CI: 71%-78%) was reduced to 75%, confirming that the specific cancer type groups were unlikely to match the respective actual cancer types.

1. A sessile droplet biosensor comprising:
a substrate;
a functional base positioned on the substrate and comprising at least one pattern; and
a bioreceptor positioned on the pattern and specifically binding to stained extracellular vesicles (EVs),
wherein a sessile droplet comprising the EVs is formed on the pattern to have a predetermined contact angle with respect to the pattern, and
an internal flow of the sessile droplet causes the EVs to migrate to the edge of the sessile droplet and specifically bind to the bioreceptor.

2. The biosensor of claim 1, wherein the contact angle is in a range of 10° to 55°.

3. The biosensor of claim 1, wherein the pattern is a perforation pattern made up of holes formed in the functional base or an uncoated pattern that is a region except for a region coated with a hydrophobic material on the substrate.

4. The biosensor of claim 1, wherein the pattern has a maximum diameter in a range of 4 mm to 10 mm.

5. The biosensor of claim 1, wherein the EVs are stained by a method that proteins or lipids in the EVs bind to a staining material.

6. The biosensor of claim 1, wherein the EVs are isolated from at least one type of patient selected from the group consisting of a cancer patient, a brain disease patient, and a cardiovascular disease patient.

7. The biosensor of claim 1, wherein the bioreceptor is at least one selected from the group consisting of an antibody, an aptamer, a nucleic acid, DNA, RNA, a biomimetic, a protein, an organic compound, and a polymer that specifically bind to the EVs.

8. A method of detecting EVs, the method comprising:
staining a sample comprising EVs;
forming a sessile droplet comprising the sample on a pattern of a sessile droplet biosensor;
incubating the sessile droplet under a predetermined humidity condition, so that the EVs specifically bind to a bioreceptor positioned on the pattern; and
detecting staining signals of the EVs specifically bound to the bioreceptor,
wherein the sessile droplet is formed on the pattern to have a predetermined contact angle with respect to the pattern, and
an internal flow of the sessile droplet causes the EVs to migrate to the edge of the sessile droplet and specifically bind to the bioreceptor.

9. The method of claim 8, wherein the contact angle is in a range of 10° to 55°.

10. The method of claim 8, wherein the staining of the sample is performed by binding proteins or lipids in the EVs to a staining material.

11. The method of claim 8, wherein the staining of the sample is performed for 30 minutes to 120 minutes.

12. The method of claim 8, wherein under the predetermined humidity condition for the incubation, a relative humidity is in a range of 20% to 90%.

13. The method of claim 8, wherein the incubation is performed in a temperature range of 20° C. to 40° C.

14. The method of claim 8, wherein the incubation is performed for 85 minutes to 95 minutes.

15. A method of analyzing staining signals of EVs, the method comprising:
obtaining a healthy domain and a cancer domain using a first result obtained through a quadratic discriminant analysis (QDA) classification algorithm from staining signals of EVs detected by the method of claim 8; and
obtaining a specific cancer domain using a second result obtained through a multiclass QDA (MultiQDA) classification algorithm from staining signals of EVs in the cancer domain.

16. The method of claim 15, wherein the obtaining of a healthy domain and a cancer domain comprises:
obtaining normalized data through principal component analysis (PCA) performed on the staining signals of the EVs; and
obtaining the healthy and cancer domains using a first result obtained through QDA performed on the normalized data.

17. The method of claim 15, wherein the obtaining of a specific cancer domain comprises:
additionally obtaining normalized data through additional PCA performed on the staining signals of the EVs in the cancer domain; and
obtaining the specific cancer domain using a second result obtained through MultiQDA performed on the additionally obtained normalized data.

18. The method of claim 15, wherein the specific cancer domain is a domain of at least one type of patient group selected from the group consisting of a lung cancer patient group, a liver cancer patient group, a breast cancer patient group, a colon cancer patient group, and a prostate cancer patient group.

19. A method of providing information to analyze staining signals of EVs, the method comprising:
obtaining staining signals of EVs in the method of claim by detecting a biological sample of an individual in need thereof using the method of claim 8;
determining whether a first result obtained through a QDA classification algorithm from the staining signals of the EVs falls within a cancer domain; and
determining a carcinoma using a second result obtained through a MultiQDA classification algorithm from the staining signals of the EVs when the first result falls within the cancer domain.
